(19) United States
(12) Patent Application Publication
(10) Pub. No.: US 2005/0032050 A1
(43) Pub. Date: Feb. 10, 2005

(54) Process for constructing dna based molecular marker for enabling selection of drought and diseases resistant germplasm screening

(76) Inventors: Villoo Morawala Patell, Secunderabad (IN); Vidya Jagannathan, Coimbatore (IN)

Correspondence Address: Sidley Austin;Brown & Wood, 1501 K Street NW, Washington, DC, 20005, US

(21) Appl. No.: 10/204,849

(22) PCT Filed: Feb. 26, 2001
(86) PCT No.: PCT/IN01/00023

(30) Foreign Application Priority Data
Feb. 25, 2000 (IN) 154/MAS/2000

(51) Int. Cl. C12Q 1/68; G06F 19/00; G01N 33/48; G01N 33/50

(52) U.S. Cl. 435/6; 702/20

(57) ABSTRACT

This invention relates to a process for constructing DNA-based molecular markers in plants comprising: identifying and selecting the gene sequences relating to stress from available database and literature; submitting the selected gene sequence for similarity search to obtain other sequences from the database similar to the selected gene sequence; subjecting the sequences obtained from similarity search to multiple alignment; removing redundant sequences if any, to get a data set of proteins involved in biotic and abiotic stress response; picking blocks or motifs from the data set of proteins on basis of statistical significance; subjecting the data set of proteins to Blockmaker to pick the same set of blocks or motifs; analysing the motifs for the functionality.

The present invention relates to a process for constructing DNA-based molecular markers in plants to detect molecular markers for various kinds stress tolerance traits in plants using a bioinformatic method.

### BACKGROUND

Plants are exposed to various adverse environmental conditions such as drought, high salt and high/low temperature etc., and to different kinds of pathogens during their life cycle. These environmental stimuli are commonly known as abiotic stress. Biotic stress on the other hand is caused by various pathogens found in the environment.

Plants respond to various kinds of stress by displaying complex, quantitative traits that involve the cumulative effect of several genes. The activation of response to any kind of stress recognition and initiation of signal transduction processes finally result in a spatially and temporally regulated gene expression.

Numerous stress inducible proteins have been identified and their corresponding genes have been isolated and sequenced. Regulatory Elements of stress-modulated genes have also been deciphered. for example Abscisic Acid Responsive Element (ABRE).

Recent developments in molecular biology and statistics along with application of information technology have opened the possibility of identifying and using genomic variation and major genes for the improvement of commercially important crops. Application of marker based selection can be more effective in characteristics that are expressed late in plants or due to certain environmental conditions or affected by few genes.

When it is not possible to distinguish plant materials visually or by simple measurements, molecular markers can sometimes be used. The Molecular markers can used to easily discern phenotypic traits. These Molecular Markers are used as a probe a mark nucleus or chromosome.

Molecular Markers may be applied for a number of purposes including determining:
Genetic identity
Parentage (maternity and paternity)
Extended kinship
Differentiation of geographic population
Differentiation of close related relationship
Phylogenetic relationship of species, family, genera, orders, phyla.
Differentiation of Populations for various genetic traits like disease resistance, drought tolerance etc.

There are two general types of molecular markers available for use depending on the plant and the type of assay required:
isoenzymes (isozymes) and
DNA-based markers
   DNA-Based Markers

DNA is the fundamental molecule of heredity consisting a double helix of linked nucleotides. DNA based Molecular markers are small sequences of DNA which are associated with or “linked” to regions in a plants DNA that are responsible for a specific trait (eg. disease resistance, yield, etc.).

There are Various Kinds of Conventional Markers Used Such as:

1. Restriction Fragment Length Polymorphism: Polymorphisms in the lengths of particular restriction fragments can be used as molecular markers. The DNA Molecule is fragmented using restriction endonuclease. Restriction endonucleases are protein enzymes that recognize specific nucleotide sequences and cleave both strands of the DNA containing those sequences.
2. Random amplified polymorphic DNA: The complexity of DNA is sufficiently high that by chance pairs of sites complementary to single octa- or decanucleotides may for amplification.
3. Microsatellites: Polymorphisms in the lengths of tandemly repeated short sequences can be used as molecular markers
4. Single-Stranded Conformation Polymorphism (SSCP): Polymorphisms in sequence, as well as in sequence length, can be used as molecular markers. The mobility in gel electrophoresis of double-stranded DNA of a given length is relatively independent of nucleotide sequence. In contrast, the mobility of single strands can vary considerably as a result of only small changes in nucleotide sequence. This fact led to the development of single-stranded conformation polymorphism (SSCP) techniques.
5. Single nucleotide Polymorphisms: Single nucleotide polymorphisms (SNP's) can be used as molecular markers.

However the conventional methods of developing markers in the laboratory is a very tedious process.

### SUMMARY OF THE INVENTION

The objective of the present invention is to correlate the occurrence of Motifs (highly conserved amino acid sequences) in various stress related proteins for molecular marker development.

Another objective is to identify a method for finding new markers from already existing sequences for the various kind of stress in plants.

Further objective is to classify these markers for the different kinds of abiotic and biotic stress the plant face.

To achieve the said objects, the present invention relates to a process for constructing DNA-based molecular markers in plants comprising:
identifying and selecting the gene sequences relating to stress from available databases and literature
submitting the selected gene sequence for similarity search to obtain other sequences from the database similar to the selected gene sequence
subjecting the sequences obtained from similarity search to multiple alignment
removing redundant sequences if any, to get a data set of proteins involved in biotic and abiotic stress response
picking blocks or motifs from the data set of proteins on basis of statistical significance
subjecting the data set of proteins to Blockmaker to pick the same set of blocks or motifs
analysing the motifs for the functionality

The invention can be used over a broad range of types of plants and organisms. Such plants inter atia includes cotton, maize, rice, soybeans, sugar beet, wheat, fruit, vegetables and vines. The major of use of the markers will be very useful to identify different varieties of plants that show stress tolerance.

The protein sequences are of length 8 and 18.

### DETAILED DESCRIPTION OF THE INVENTION WITH THE ACCOMPANYING FIGURES

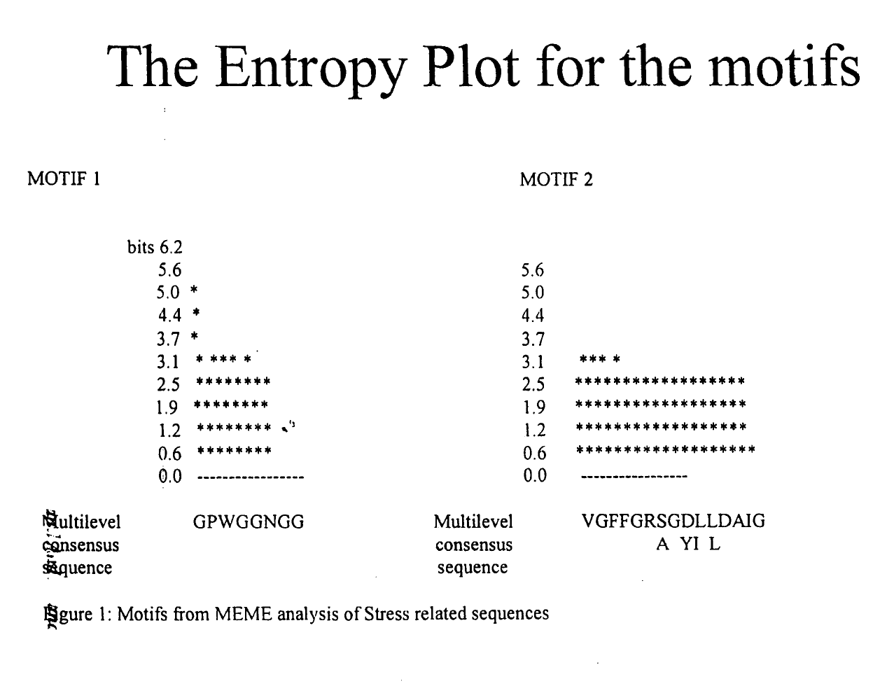

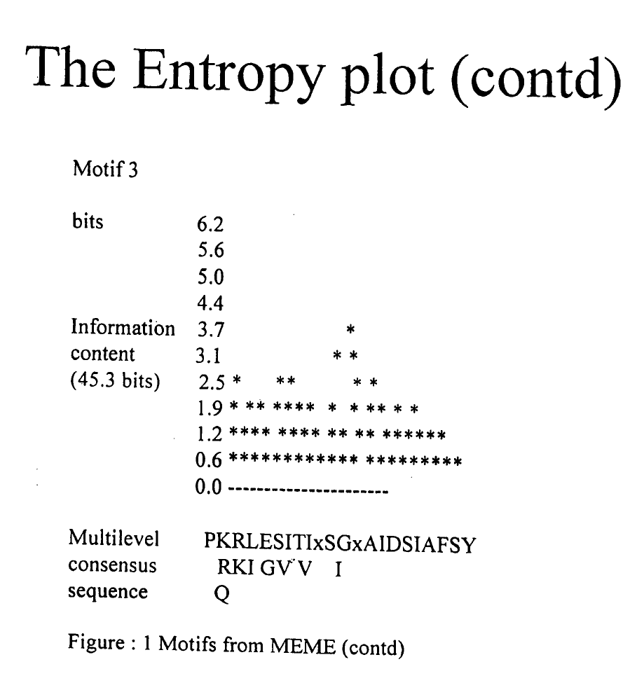

FIG. 1 displays the three motifs of the stress dataset along with the entropy plot, which is the measure of the information content at each position.

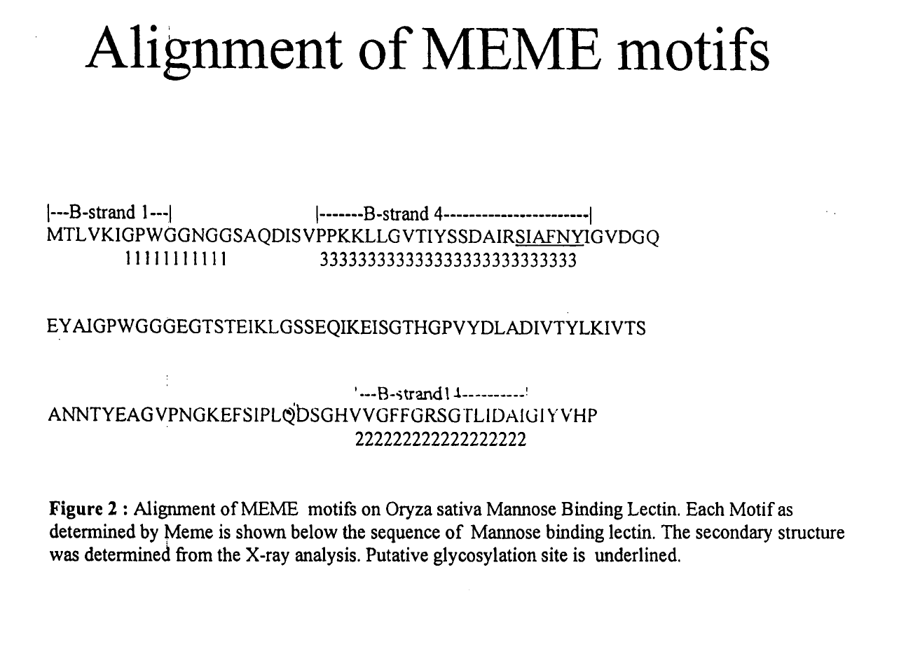

FIG. 2 shows the motifs are mapped on to the Mannose binding letcin

Table 1 shows the sequences details with their Swissprot codes.

Table 2 shows the details of the evaluation of the first motif.

A Sequence analysis of stress related sequences, was done as follows:

Stress related sequences were downloaded from Swissprot and the PIR databases and a literature study of the sequences were carried out to pick a protein, which was well characterized experimentally to be involved in stress.

The salT gene of Oryza sativa was selected for further studies.

### EXAMPLE 1

The salT protein was submitted for similarity search and around 65 proteins were obtained. 15 proteins were selected based on the threshold of 35% similarity and the set was reduced to 12 after removing the redundant sequences. The data set of the twelve sequences consisted of proteins involved in various biotic and abiotic stress responses.

An analysis was conducted to discover potential regions of sequence homology between twelve biotic and abiotic stress-related genes. The homology analysis resulted in 3 non-overlapping motifs that were common to both biotic and abiotic stress-related genes.

A total of 113 new genes were identified. The annotation present for each of the genes supports the hypothesis that they are involved in stress-related response.

Multiple Alignment and Statistical Significance

The length of sequences used for making the blocks or motifs are varied and the motifs do not occur in a specific position in all these sequences. Besides, since the proteins are made up of only 20 amino acids, a statistical analysis is done to check whether the identified motif has occurred by chance, or whether its presence in the sequence is of any significance.

The end result is of the probability of occurrence is as follows:
a. if the occurrence of this pattern is high then it is of no significance,
b. it the probability of occurrence is very low, then this probability has also a biological significance.

The twelve sequences were then subjected to multiple alignment using clustalW. Three non-overlapping motifs were picked up manually by ‘eye’. The statistical significance of blocks of similarity was evaluated using the MACAW (Multiple Alignment Construction and Analysis Workbench)

The same data set was submitted to Blockmaker and analysed for the presence of Blocks. The same sets of blocks were picked up by the Program.

Analysis of Motifs using MEME (Multiple Expectation Maximization for Motif Elcitation). The three strongest motifs in the set of 12 sequences of twelve divergent sequences were determined using MEME 2.0.

These motifs were used to generate a Position Specific Scoring Matrix (PSSM) in order to identify further stress-related genes from the public sequence databases. The Position Specific Scoring Matrix of the MEME output was then used to search the Genbank and Swissprot 39.4 using the MAST (Motif Alignment and search tool)

The three motifs map on to functionally important domains. The first motif relates to a common epitope and the third motif maps on to an important N-glycosylation site.

Motif Listing:

| | | |
|---|---|---|
| 1 | 18 | VITSLTFKTNKKTYGPFG |
| 2 | 8 | GPWGGNGG |
| 3 | 16 | IVGFFGRSGWYLDAIG |

### REFERENCES

1. Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680.
2. Schuler, G. D., Altschul, S. F, Lipian, D. J. (1991) A workbench for multiple alignment construction and analysis. Proteins: Structure, Function and Genetics 9:180-190.
3. http://blocks.fhcrc.org/blocks/blockmkr/make_blocks.html
4. Henikoff, S., Henikoff, J. G, Alford, W. J, and Pietrokovski, S. (1995), Automated construction and graphical presentation of protein blocks from unaligned sequences, Gene 163:GC17-26.
5. Timothy L Bailey and Charles Ellkan, “Fitting a mixture model by expectation maximization to discover motifs in biopolymers”, Proceedings of the Second International Conference on Intelligent Systems for Molecular Biology, pp. 28-36, AAAI Press, Menlo Park, Calif., 1994.
6. http://meme.sdsc.edu/meme/website/mast.html
7. Timothy L Bailey and Michael Gribskov, “Combining evidence using pvalues: application to sequence homology searches”, Bioinformatics, 14:1, pp. 48-54.
8. Tsuda. M (1979) Purification and characterisation of a lectin from rice. J. Biochem. 86: 1451-1461

9. Ko Hirano, Tohru Teraoka, Homare Yamanaka, Akane Harashima, Aldko Kunisaki, Rideki Takashi and Daiiro Hosokawa Novel Mannose-Binding Rice Lectin Composed of some Isolectins and its relation to a Stress-Inducible salT Gene, Plant Cell Physiol. 41(3): 258-267 (2000)

TABLE 1
The 12 Sequences with their Swissprot codes.

| SWISSPROT IDENTIFIER | DESCRIPTIONS |
|---|---|
| SALT_ORYSA | Salt resistance gene of Oryza |
| O64441 | Mannose binding lectin of Oryza |
| O04184 | Oryza SalT mma |
| GOS9_ORYSA | Root specific stress realated gene |
| Q40007 | Jasmonate induces protein |
| Q9xG950 | Light stress protein in barley |
| Q41519 | Benzothiadozole induced disease resistance |
| | associated protein |
| 080370 | Vernalisation related protein |
| Q9ZOyY4 | Lectin 17 |
| AF232008 | Beta galactosidase aggregate (heat shock |
| | protein) |
| AAD11578 | Helinathus annus -lectin (mannose binding) |
| A58801 | Mannose specific lectin of Jack Fruit |

| Sequence Name | Description | E-value | Length |
|---|---|---|---|
| gb\|AF064032.1\|AF064032 | Helianthus tuberosus | 1.4e−30 | 552 |
| | lectin HE1 . . . | | |
| gb\|AF064031.1\|AF064031 | Helianthus tuberosus | 2.7e−30 | 675 |
| | lectin 3 m . . . | | |
| gb\|AF064029.1\|AF064029 | Helianthus tuberosus | 4.3e−30 | 779 |
| | lectin 1 m . . . | | |
| gb\|AF064030.1\|AF064030 | Helianthus tuberosus | 5.2e−30 | 829 |
| | lectin 2 m . . . | | |
| gb\|U43497.1\|HVU43497 | Hordeum vulgare | 1.6e−29 | 1091 |
| | putative 32.7 k . . . | | |
| gb\|AF021257.1\|AF021257 | Hordeum vulgare | 1.1e−27 | 4487 |
| | 32 kDa protein . . . | | |
| gb\|U43496.1\|HVU43496 | Hordeum vulgare | 1.2e−27 | 1505 |
| | putative 32.6 k . . . | | |
| gb\|AF021256.1\|AF021256 | Hordeum vulgare | 1.9e−26 | 3786 |
| | 32 kDa protein . . . | | |
| dbj\|D89823.1\|D89823 | Ipomoea batatas | 3.7e−26 | 720 |
| | mRNA for ipomoe . . . | | |
| gb\|U56820.1\|CSU56820 | Calystegia sepium | 4.5e−24 | 714 |
| | lectin mRNA, . . . | | |
| gb\|AF232008.1\|AF232008 | Zea mays beta- | 4.2e−23 | 1087 |
| | glucosidase aggre . . . | | |
| gb\|AF001527.2\|AF001527 | Musa acuminata | 1.3e−22 | 705 |
| | ripening-associa . . . | | |
| gb\|AF021258.1\|AF021258 | Hordeum vulgare | 4.4e−22 | 1792 |
| | 32 kDa protein . . . | | |
| dbj\|D85194.1\|D85194 | Arabidopsis thaliana | 1.6e−21 | 2200 |
| | mRNA, part . . . | | |
| gb\|AF222537.1\|AF222537 | Arabidopsis thaliana | 2.2e−21 | 2461 |
| | myrosinase . . . | | |
| dbj\|AB027252.1\|AB027252 | Arabidopsis thaliana | 2.2e−21 | 2464 |
| | gene for f . . . | | |
| emb\|Y11482.1\|BNJIP3133 | B. napus mRNA for | 2e−20 | 3133 |
| | jasmonate indu . . . | | |
| emb\|Y09437.1\|BNMYBIPRO | B. napus mRNA for | 2.1e−20 | 3200 |
| | myrosinase bin . . . | | |
| dbj\|AB032412.1\|AB032412 | Arabidopsis thaliana | 2.7e−20 | 5719 |
| | f-AtMBP ge . . . | | |
| gb\|AC008017.2\|AC008017 | Arabidopsis thaliana | 4.7e−18 | 116944 |
| | chromosome . . . | | |
| gb\|U32427.1\|TAU32427 | Triticum aestivum | 6.5e−18 | 1209 |
| | clone WCI-1 u . . . | | |
| emb\|AJ237754.1\|HVU237745 | Hordeum vulgare high | 3.3e−17 | 623 |
| | light-indu . . . | | |
| gb\|U59443.1\|BNU59443 | Brassica napus | 3.5e−17 | 3173 |
| | myrosinase-bindi . . . | | |
| gb\|AC006216.1\|F5F19 | Arabidopsis thaliana | 1.5e−16 | 110893 |
| | chromosome . . . | | |
| gb\|AF054906.1\|AF054906 | Arabidopsis thaliana | 5.5e−15 | 1629 |
| | myrosinase . . . | | |
| gb\|L03798.1\|ARPJACD | Artocarpus integrifolia | 6.5e−14 | 845 |
| | jacalin . . . | | |
| gb\|L03796.1\|ARPJACB | Artocarpus integrifolia | 7.1e−14 | 871 |
| | jacalin . . . | | |
| dbj\|AP000373.1\|AP000373 | Arabidopsis thaliana | 7.2e−14 | 71521 |
| | genomic DN . . . | | |
| gb\|AC001645.1\|ATAC001645 | Arabidopsis thaliana | 1.5e−13 | 91714 |
| | chromosome . . . | | |
| gb\|L03795.1\|ARPJACA | pSKcJA1; Artocarpus | 2.1e−13 | 846 |
| | integrifoli . . . | | |
| gb\|L03797.1\|ARPJACC | Artocarpus integrifolia | 3.1e−13 | 846 |
| | jacalin . . . | | |
| gb\|AC024609.2\|AC024609 | F14P1, complete | 7.4e−13 | 90341 |
| | sequence [Arabi . . . | | |
| gb\|AC007797.7\|AC007797 | Arabidopsis thaliana | 1.7e−12 | 119942 |
| | chromosome . . . | | |
| gb\|AF001395.1\|OSAF001395 | Oryza sativa salT | 1.7e−12 | 631 |
| | mRNA, complet . . . | | |
| dbj\|AB012605.1\|AB012605 | Oryza sativa gene for | 9.8e−12 | 1139 |
| | MRL, comp . . . | | |
| emb\|Y11483.1\|BNJIP2268 | B. napus mRNA for | 1e−11 | 2268 |
| | jasmonate indu . . . | | |
| gb\|AF214573.1\|AF214573 | Arabidopsis thaliana | 7.5e−11 | 1177 |
| | myrosinase . . . | | |
| gb\|S45168.1\|S45168 | salT = 15 kda organ- | 1.6e−10 | 724 |
| | specific salt . . . | | |
| dbj\|AB012103.2\|AB012103 | Triticum aestivum | 8.9e−10 | 1563 |
| | mRNA for VER2 . . . | | |
| emb\|X51909.1\|OSGOS9G | O. sativa (rice) root- | 1.2e−09 | 3350 |
| | specific . . . | | |
| emb\|Z25811.1\|OSSALT | O. sativa salT gene | 6.3e−09 | 2637 |
| gb\|U59444.1\|BNU59444 | Brassica napus | 3.8e−08 | 2176 |
| | myrosinase-bindi . . . | | |
| gb\|AC004697.2\|AC004697 | Arabidopsis thaliana | 5.6e−08 | 106718 |
| | chromosome . . . | | |
| gb\|AC010164.2\|AC010164 | Arabidopsis thaliana | 7.4e−08 | 103443 |
| | chromosome . . . | | |
| dbj\|AB026643.1\|AB026643 | Arabidopsis thaliana | 1.2e−07 | 84710 |
| | genomic DN . . . | | |
| gb\|U59446.1\|BNU59446 | Brassica napus | 2.3e−07 | 1923 |
| | myrosinase-bindi . . . | | |
| gb\|U59445.1\|BNU59445 | Brassica napus | 4.1e−07 | 1751 |
| | myrosinase-bindi . . . | | |
| gb\|AC004473.1\|T13D8 | Arabidopsis thaliana | 8e−06 | 116177 |
| | chromosome . . . | | |
| dbj\|AP000373.1\|AP000373 | Arabidopsis thaliana | 0.00016 | 71521 |
| | genomic DN . . . | | |
| gb\|AC004747.2\|AC004747 | Arabidopsis thaliana | 0.00016 | 80283 |
| | chromosome . . . | | |
| gb\|AC001645.1\|ATAC001645 | Arabidopsis thaliana | 0.00027 | 91714 |
| | chromosome . . . | | |

1. A process for constructing DNA-based molecular markers in plants comprising:
identifying and selecting the gene sequences relating to stress from available databases and literature
submitting the selected gene sequence for similarity search to obtain other sequences from the database similar to the selected gene sequence
subjecting the sequences obtained from similarity search to multiple alignment
removing redundant sequences if any, to get a data set of proteins involved in biotic and abiotic stress response
picking blocks or motifs from the data set of proteins on basis of statistical significance
subjecting the data set of proteins to Blockmaker to pick the same set of blocks or motifs
analysing the motifs for the functionality

2. A process for constructing molecular markers as claimed in claim 1 wherein the gene selected is that of Oryza sativa

3. A process for constructing molecular markers as claimed in claim 1 wherein the database used is Swissprot and PIR

4. A process for constructing molecular markers as claimed in claim 1 wherein the software used to subject the sequences to multiple alignment is clustalW

5. A process for constructing molecular markers as claimed in claim 1 wherein the software used to conduct the similarity search is Multiple Alignment Construction and Analysis Workbench (MACAW)

6. A process for constructing molecular markers as claimed in claim 1 wherein the software used for marking blocks are the Blockmakers

7. A process for constructing molecular markers as claimed in claim 1 wherein the motifs are analyzed using Multiple Expectation Maximization for Motif Elicitation (MEME)

8. A process for constructing molecular markers as claimed in claim 1 wherein the amino acid sequence or the motif in the isolated protein sequences are 8 to 18

9. A process for constructing molecular markers as claimed in claim 1 wherein the motif 1 is VITSLTFKTNKKTYGPFG

10. A process for constructing molecular markers as claimed in claim 1 wherein the motif 2 is GPWGGNGG

11. A process for constructing molecular markers as claimed in claim 1 wherein the motif 3 is IVGFFGRSGWYLDAIG

12. A process for constructing molecular markers as claimed in claim 9 wherein the motif 1 relates to a common epitope.

13. A process for constructing molecular markers as claimed in claim 11 wherein the motif 3 maps an important n-glycosylation site
